(19) United States
(12) Patent Application Publication
(10) Pub. No.: US 2024/0240359 A1
(43) Pub. Date: Jul. 18, 2024

(54) BIOCATALYTIC PLATFORM FOR CHEMICAL SYNTHESIS

(71) Applicant: THE REGENTS OF THE UNIVERSITY OF MICHIGAN, Ann Arbor, MI (US)

(72) Inventor: Alison R. H. Narayan, Ann Arbor, MI (US)

(21) Appl. No.: 18/268,538

(22) PCT Filed: Dec. 22, 2021
(86) PCT No.: PCT/US2021/064790

§ 371 Date: Jun. 20, 2023

Related U.S. Application Data
(60) Provisional application No. 63/129,003, filed on Dec. 22, 2020.

(51) Int. Cl. C40B 40/08; C12P 1/00

(52) U.S. Cl. CPC C40B 40/08; C12P 1/00

(57) ABSTRACT

Disclosed herein are methods for synthesizing or functionalizing organic compounds using a library of biocatalysts. The methods include separately admixing a reactant and an aqueous solvent with each biocatalyst in a library of biocatalysts to provide a library of product admixtures, wherein the admixing occurs under sustainable reaction conditions.

### STATEMENT OF GOVERNMENT INTEREST

This invention was made with government support under GM124880 awarded by the National Institutes of Health. The government has certain rights in the invention.

### FIELD OF THE INVENTION

The disclosure generally provides methods of preparing organic compounds. More specifically, the disclosure provides methods of preparing organic compounds using a library of biocatalysts.

### BACKGROUND

The discovery of new small molecules, e.g., for use as drugs, is labor intensive and can be rate limiting, requiring large teams of chemists working for weeks or months to generate the new structures. As a result, (1) the timeline for drug discovery is slowed and (2) the number of compounds generated and the complexity of these molecules is directly tied to the resources available.

Currently, modern medicines and biological probes are prepared through the combination of small molecule reagents and catalysts in an iterative fashion. Additionally, many synthetic pathways utilize reactants and/or solvents that will destroy a biological system. Thus, isolation and purification of target molecules is required, which only adds to the labor and time costs of developing new molecules for use in biological systems.

Thus, there is a need for methods for increasing the speed and precision with which new complex molecules can be synthesized and for methods where the conditions of synthesis are compatible with biological systems and physiological conditions.

### SUMMARY

One aspect of the disclosure provides methods for synthesizing organic compounds comprising: separately admixing a first reactant and an aqueous solvent with each biocatalyst in a library of biocatalysts to provide a library of product admixtures, wherein the admixing occurs under sustainable reaction conditions, and each product admixture comprises: (i) a first product formed from a chemical reaction between the first reactant and each biocatalyst, (ii) the aqueous solvent, and (iii) the biocatalyst. Optionally, the methods can further comprise admixing a second reactant in situ with one or more product admixtures in the library of product admixtures, wherein the second reactant reacts with the first product in the one or more product admixtures to form a second product. Optionally, the methods can further comprise subjecting one or more of the first products to one or more biological assays without isolating the one or more first products from the one or more product admixtures.

Another aspect of the disclosure provides methods of diversifying a biologically active molecule comprising: separately admixing the biologically active molecule and an aqueous solvent with each biocatalyst in a library of biocatalysts to provide a library of biologically active product admixtures, wherein the admixing occurs under sustainable reaction conditions, and each biologically active product admixture comprises: (i) a first biological product formed from a chemical reaction between the biologically active molecule and each biocatalyst, (ii) the aqueous solvent, and (iii) the biocatalyst.

Further aspects and advantages will be apparent to those of ordinary skill in the art from a review of the following detailed description. While the methods disclosed herein are susceptible of embodiments in various forms, the description hereafter includes specific embodiments with the understanding that the disclosure is illustrative, and is not intended to limit the invention to the specific embodiments described herein.

### BRIEF DESCRIPTION OF THE DRAWINGS

FIG. 1 depicts representative end products of the high throughput drug discovery enabled by biocatalysis using the methods disclosed herein. Protein collections generated are profiled over chemically diverse substrate libraries for late-stage functionalization (left) and building molecular cores chemoenzymatically and through total biocatalytic routes (right).

FIG. 2A depicts the workflow for high-throughput biocatalytic reactions of lead compounds with the enzyme libraries having the potential to couple enzymatic reactions in well plates directly with biological assays that define structure-activity relationship using the methods herein.

FIG. 2B depicts late-stage modifications, including hydroxylation, halogenation, methylation and fluoroalkylation using the methods disclosed herein.

FIG. 2C is an example of classes of enzymes that libraries can be built around using the methods disclosed herein.

FIG. 3 depicts three different approaches for accessing diverse enzyme catalysts using the methods disclosed herein: enzymes from natural product gene clusters (left), family-wide activity profiling (center), and protein engineering (right).

FIG. 4 depicts a sequence similarity network (SSN) of α-KG-dependent NHI dioxygenase protein family that highlights clustering of function and diversity of substrate scope across families. The SSN shows a cluster of enzymes that carry out benzylic hydroxylation (left), a cluster of enzymes that mediates oxidative ring-expansion (center), and a sequence space with yet uncharacterized function (right).

FIG. 5A depicts a section of a sequence similarity network (SSN) containing CitB and ClaD as well as 168 related sequences for use in the methods disclosed herein.

FIG. 5B depicts the hydroxylation activity of an NHI enzyme library.

FIG. 5C depicts substrates hydroxylated by at least one enzyme in an NHI library prepared from a SSN containing CitB and ClaD, which were not substrates for hydroxylation by CitB and ClaD using the methods disclosed herein.

FIG. 6A depicts conserved oxidative dearomatization activity across the first-generation flavin-dependent monooxygenase sequence-diverse library, using the methods disclosed herein.

FIG. 6B depicts the presence of stereocomplementary catalysts within the first-generation flavin-dependent monooxygenase sequence-diverse library, using the methods disclosed herein.

FIG. 6C depicts the need for substantial libraries of enzymes to capture broad substrate scope within the first-generation flavin-dependent monooxygenase sequence-diverse library, using the methods disclosed herein.

FIG. 7 depicts strategies for building molecular frameworks using biocatalysis by the methods disclosed herein, the strategies include: biocatalytic generation of reactive intermediates that are readily intercepted by small molecule reagents, including enzymatic benzylic hydroxylation initiated ortho-quinone methide formation and enzymatic oxidative dearomatization to generate reactive dienone species (left), and convergent biocatalysis for assembly of biaryl C—C bonds (right).

FIG. 8 depicts data demonstrating the biocatalytic C—C bond formation in oxidative cross coupling using the methods disclosed herein.

FIG. 9 depicts a substrate scope analysis for CitB and ClaD mediated oxidation using the methods of the disclosure.

FIG. 10 depicts a sequence similarity network of NHI-dependent monooxygenases related to CitB and ClaD.

FIG. 11 depicts NHI libraries exploited for the chemoenzymatic synthesis of structurally diverse natural products according to methods of the disclosure.

FIG. 12 depicts substrate promiscuity of flaming-dependent monooxygenase involved in fungal secondary metabolism using methods of the disclosure.

FIG. 13 depicts biosynthetic cytochromases P450 (1-12) and laccases (13-16) known to catalyze oxidative dimerization reactions to form biaryl products and the related sequence similarity network (SSN) analysis showing a much broader pool of related, underexplored natural sequence.

FIG. 14A depicts the Fungal P450 KtnC mediated cross-coupling of coumarins with high conversion using methods of the disclosure.

FIG. 14B depicts the CYP158A2 mediated cross-coupling of naphthols with moderate to high conversion using methods of the disclosure.

FIG. 15 depicts multi site-directed mutagenesis and site-saturation mutagenesis of fungal P450 KtnC for identifying a variant having 12.5-fold improvement in conversion for non-native cross-coupling reactions.

### DETAILED DESCRIPTION

Disclosed herein are methods of preparing organic compounds using a library of biocatalysts, wherein the organic compounds are prepared with increased speed and precision relative to the current state of the art.

Satisfying the demand for new and increasingly complex molecules requires a step-change in the speed and precision with which new complex molecules are synthesized. In Nature's approach to building molecules, hundreds of different enzymes carry out their individual chemical reactions simultaneously in a single cell. By harnessing Nature's catalysts to access many different classes of molecules and to diversify lead compounds through late-stage functionalization, a platform has been developed for rapid chemical synthesis wherein thousands of reactions can be run simultaneously using miniaturized experimentation and robotics. The step-change merges miniaturized high-throughput experimentation technology with the world's largest library of biocatalysts. Put simply, the methods of the disclosure make molecules better and faster by merging robotic machinery with Nature's machinery. Ultimately, one effect of the disclosed methods is to accelerate the discovery of lifesaving drugs that will impact the treatment protocols and outcomes for patients.

The methods of the disclosure introduce a paradigm shift in how molecules are assembled and diversified. This platform is also designed to minimize the environmental footprint of synthetic chemistry. Biocatalytic chemistry is highly sustainable as it relies on catalysts made from renewable feedstocks that degrade into benign byproducts. The use of enzymes as catalysts avoids toxic and hazardous reagents, such as heavy metals and environmentally detrimental solvents, required for traditional chemistry, enabling the sustainable synthesis of drugs.

The diversification of biologically active molecules with a library of biocatalysts has been explored, and the efficiency of multi-enzyme cascades in a medium-throughput fashion has been evaluated. Importantly, the results provided herein indicate that the disclosed approach can be efficiently applied to various molecule classes. In addition, protein engineering has been used for a spectrum of protein families, including, but not limited to pyridoxal phosphate (PLP)-dependent enzymes, cytochromes P450 and non-heme, iron-dependent enzymes. Specifically, site-saturation mutagenesis, combinatorial site-saturation mutagenesis and, at times, error-prone PCR is employed for library generation.

The discovery of new small molecule drugs is labor intensive. Often this process is initiated by evaluating a library of compounds against a specific biological target to identify structures as a starting point for iterative cycles of improving the next generation of molecules. Specifically, small changes to the structure of the lead compound are designed, the new molecules are synthesized and their properties are assessed in a design-build-test cycle. Importantly, the molecules that can be access are dictated by the methods available and the time it takes to employ developed synthetic strategies.

The methods of the disclosure focus on identifying and leveraging the enzymes evolved by Nature for producing remarkably complex secondary metabolites.¹⁻¹¹ These molecules, made by live organisms, are famous for their diverse structures and potent biological activities, making up over half of all antibiotics and cancer drugs.¹² The chemical space accessible through enzyme chemistry is vast, yet has not traditionally been accessible to synthetic chemists. The methods of the disclosure advantageously remove this barrier and bring enzymes to the chemist's bench using a library of biocatalysts unprecedented in size and diversity.

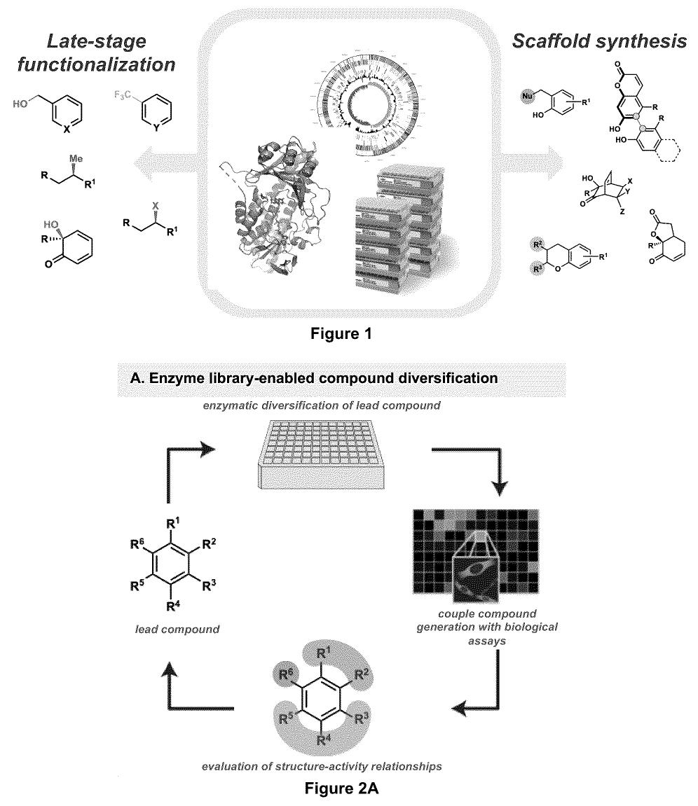

The methods of the disclosure leverage (a) accessing complex molecules through biocatalytic late-stage functionalization,^{8, 10} (b) building enzyme libraries that will enable transformations on a breadth of substrates^{5, 9} and (c) demonstrating the power of applying biocatalytic retrosynthetic logic in the design of synthetic routes that can be adapted to the high throughput generation of compound libraries.^{2, 5} Pilot libraries of natural and engineered enzymes have been built that span flavin-dependent monooxygenases, non-heme iron-dependent enzymes, methyltransferases, acyltransferases and C—C bond forming cytochrome P450s. The reactivity and selectivity of known enzymes have been profiled against known structures. The methods of the disclosure can facilitate expansion of these efforts to include compound collections and additional target scaffolds (FIG. 1). The magnitude of compounds and target scaffolds that can be accessed by the methods disclosed herein is significant and can be enabled by ultra-high throughput mass spectrometry methods. The resulting data can identify transformations and substrate classes that motivate the further expansion of the enzyme library to better facilitate the high throughput molecule generation for the purpose of drug discovery.

### Biocatalytic Diversification for High Throughput Compound Generation

Biocatalysis is routinely employed in process chemistry routes, where an enzyme is often engineered to operate with the high efficiency and precise selectivity required for a manufacturing route. This requires a substantial investment to develop a single biocatalytic step.¹³ However, when this level of perfection is not required, the barrier to incorporating biocatalysis into synthesis is much lower. For example, wild type enzymes often do not need to be trained to act on non-native substrates.^{2, 9} By embracing the inherent substrate promiscuity that is common to enzymes involved in secondary metabolism, molecules can be biocatalytically transformed without the need for protein engineering. Given this substrate flexibility, the advantages of biocatalysis can be brought into the discovery chemistry workflow to diversify scaffolds of interest with chemo-, site- and stereoselectivity only possible with enzymes. The advantages of biocatalysis for late-stage modification are significant, including: (1) chemoselectivity avoiding the need for protecting groups, (2) catalyst-controlled site-selectivity, and/or (3) amenability to analytical-scale reactions in plates minimizing material required for diversification efforts. This strategy is appropriate for any type of reaction which can be mediated enzymatically, or those which can be envisioned, including, but not limited to late-stage oxygenation, halogenation, methylation and fluoroalkylation.

Enzyme libraries suitable for, for example, late-stage hydroxylation, halogenation, methylation and fluoroalkylation on a breadth of substrates facilitate the high throughput late-stage modification of lead compounds in a format that can be directly coupled with, for example, biological assays. Advantageously, because the late-stage modification of the lead compounds is done under biologically compatible conditions, the resulting product compounds can be used without requiring isolation or purification.

Biocatalysis can provide methods that are complementary to existing small molecule methods while offering selective, sustainable and relatively safe reaction conditions.¹⁴⁻¹⁵ However, for biocatalysis to occupy space in mainstream organic synthesis, a greater breadth of well-developed biocatalytic tools is needed.¹⁶ Key challenges hindering the application of biocatalysts in synthesis include (1) the identification of enzymes capable of catalyzing a desired reaction on a target substrate and (2) developing strategies for integration of biocatalysis into synthetic sequences. Herein, a strategy is introduced for profiling the chemistry across families of enzymes to identify biocatalysts which display suitable reactivity, possess complementary substrate scope activities and demonstrated scalability to enable target-oriented chemoenzymatic synthesis.

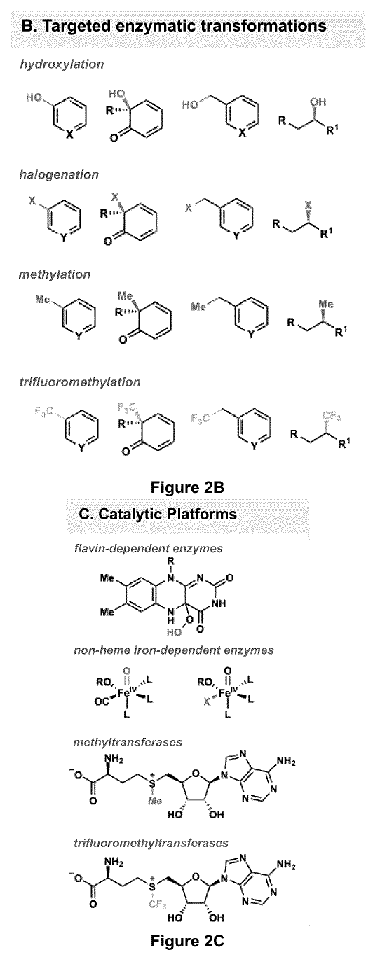

The discovery of new biocatalysts can enable the efficient diversification of complex molecules.¹⁷ However, identifying a biocatalyst that performs a desired reaction on a specific can be a challenge. It has been found that reactivity profiling of enzymes beyond those associated with known biosynthetic gene clusters can provide panels of robust, selective biocatalysts that together possess an expanded substrate scope.¹⁸ The availability of this type of enzyme panel makes biocatalysis a viable approach to late-stage diversification of compounds integral to drug discovery campaigns without the immediate need for protein engineering, which can require skills and investment beyond what is typically available to academic and industrial organic chemists (FIG. 2A).

As a starting point for expanding the number of well-characterized catalysts available to chemists, enzymes that are catalytically robust, proven on preparative-scale, and which provide a platform to achieve reactivity and selectivity that complements established small molecule methods were focused on. In addition, enzyme libraries capable of reactions that are value-added late-stage modifications were built on. In embodiments, panels deliver catalysts for (a) aromatic and alkyl hydroxylation, (b) aromatic and alkyl halogenation, (c) methylation, and (d) trifluoroalkylation (FIG. 2B). Contemplated classes of enzymes for development include (a) flavin-dependent monooxygenases naturally known to hydroxylate and halogenate substrates with high-levels of chemo-, site- and stereoselectivity (b) non-heme iron-dependent (NHI) dioxygenases that use α-ketoglutarate (α-KG) as a co-substrate paired with molecular oxygen to arrive at the active Fe(IV)-oxo species that commonly initiates reactions through hydrogen atom abstraction with exquisite control over site- and stereoselectivity that can facilitate hydroxylation and halogenation among other fates of the substrate centered radical,¹⁹⁻²⁰ and (c) methyltransferases that can selectively alkylate with natural or unnatural cofactors (FIG. 2C).

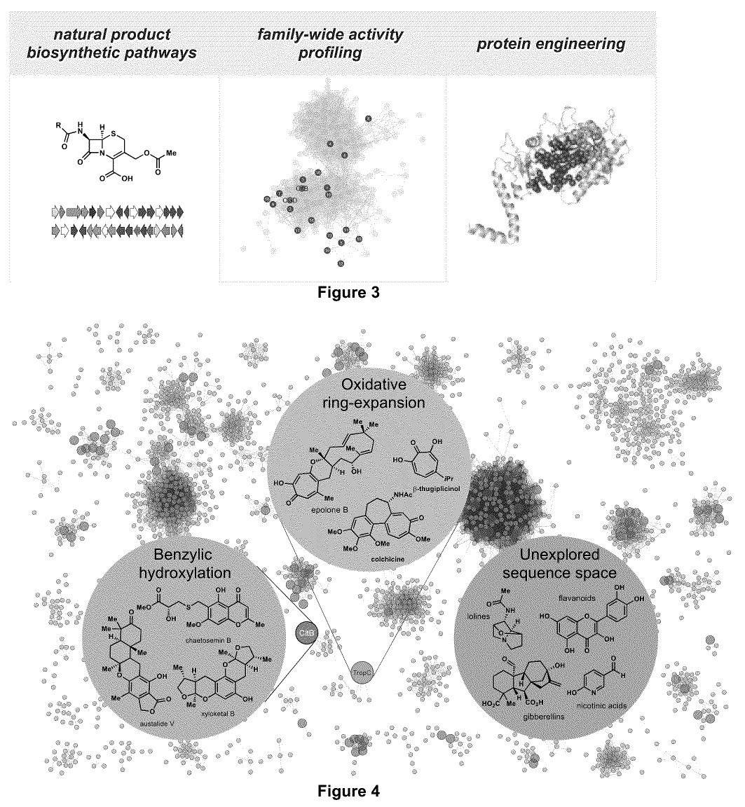

Advances in sequencing and bioinformatic tools continue to accelerate the discovery of new enzymes. For example, the number of annotated sequences for NHI biocatalysts has grown exponentially over the last decade. At the same time, the application of NHI enzymes applied in synthesis has not increased proportionately.²¹ Based on an analysis of the >100,000 known sequences in this enzyme class, the native substrate and chemical function of <1% of these enzymes is known. Within the minuscule set of enzymes with characterized chemistry, function is most commonly discovered in the context of natural product biosynthetic pathways, which provide context for the type of substrate and chemistry associated with a given enzyme (FIG. 3). These characterized biosynthetic enzymes provide footholds for exploring the substrate promiscuity and synthetic utility of these catalysts and related sequence space for which the context of a natural product biosynthetic gene cluster is not provided (FIG. 3, center). In embodiments, the methods of the disclosure are built on studies with NHI oxygenases and flavin-dependent monooxygenases involved in natural product biosynthesis²²⁻²⁴ as well as pilot libraries assembled through a family-wide profiling approach. These biocatalysts can be used to (1) explore the utility of these catalysts in the late-stage modification of structurally diverse substrates and (2) to navigate through sequence space and identify additional catalysts with synthetic value.

Bioinformatic analysis of protein families through the construction of phylogenetic trees, sequence similarity networks (SSNs),^{18, 25-26} and VAE latent space analysis²⁷ can inform the selection of protein sequences that span sequence space across each targeted protein family (e.g., flavin-dependent monooxygenases, NHI-dependent dioxygenases, and methyltransferases). For example, an NHI library can include sequences from each cluster of the SSN shown in FIG. 4—benzylic hydroxylation enzymes, oxidative ring-expansion enzymes, and enzymes with yet uncharacterized functions. First-generation libraries for each enzyme class include ˜1,000 enzymes from each family. Plasmids containing synthesized genes can be used to transform competent BL21 E. coli cells in 96 well plate format. Protein level in expression cultures can be assessed through SDS PAGE analysis and optimized accordingly by varying time, temperature, ITPG concentration, media, and cell line. Liquid handling robots can be used for high throughput gel electrophoresis. Shaking incubators are specifically suited for high rpm shaking of well plates and can provide superior aeration and increased capacity for library work in plates.

Each enzyme panel can be profiled for substrate scope, selectivity, and reaction promiscuity. This can define the structural features of compounds successfully functionalized. First-generation libraries can be profiled for reactivity and substrate promiscuity against a panel of diverse substrates. The substrate panels can contain a collection of commercially available compounds as well as synthesized, non-commercially available molecules. Reactions can be conducted in 96 and 384 well plates with total reaction volumes ranging from 25-250 μL. Standard reactions contain 1-100 mM substrate, clarified cell lysate, necessary cofactors and buffer. Reaction outcome can be assessed by UPLC, UPLC-MS, and/or RapidFire-MS. Raw data can be processed using Agilent software. Reactivity data is also analyzed for trends and fed to machine learning platforms to inform the sequences included in second-generation libraries (e.g., Scaffold Hunter, MOE, Schrodinger). This profiling will define the substrate scope covered by the library as well as illustrate scaffolds that require expansion of the enzyme library.

As described in the examples, below, libraries for two enzyme classes for late-stage functionalization platforms have been built: α-KG-dependent NHI dioxygenases and flavin-dependent monooxygenases. Toward demonstrating the synthetic utility of α-KG-dependent NHI oxygenases, experiments were initiated with two dioxygenases associated with natural product biosynthesis, CitB and ClaD. CitB and ClaD each are known to perform chemo- and site-selective C—H hydroxylation of a benzylic methyl group within a polyketide synthase-derived resorcinol compound in cintrinin and peniphenone D biosynthetic pathways, respectively.²³⁻²⁴ This transformation is deceptively challenging using traditional methods, as over-oxidation and poor chemo- and/or site-selectivity often generate undesired products or complex mixtures of products.²⁸ Common synthetic methods include transition-metal catalyzed oxidations with iron,²⁹ cobalt,³⁰ iridium,³¹ copper^{28, 32} and manganese^{30, 33} as well as heterogeneous catalysis (Au/Pd catalyst).³⁴ These methods are often plagued by low site-selectivity, over-oxidation and low functional group tolerance, requiring protecting groups to avoid side-reactions. In contrast to the traditional methods, the disclosed methods can use α-KG-dependent NHI oxygenases to provide a functional group-tolerant, catalytic and site-selective method to directly access highly substituted benzylic alcohols without over-oxidation. As described in the examples, CitB and ClaD activity with a range of substrates was determined, revealing complementarity in substrate scope of the two enzymes.

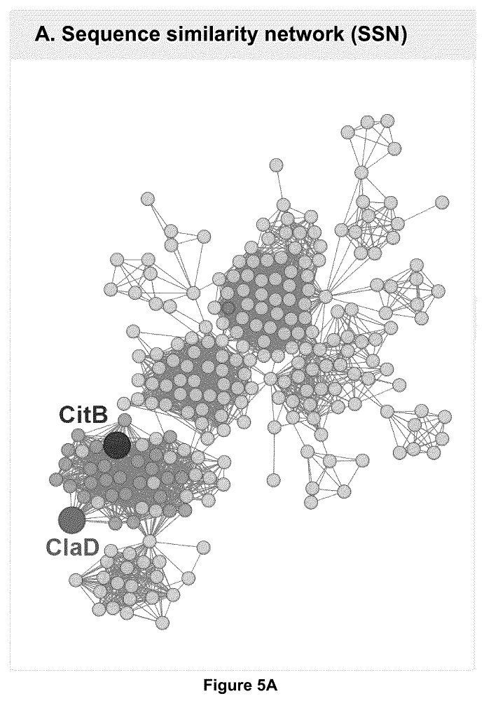

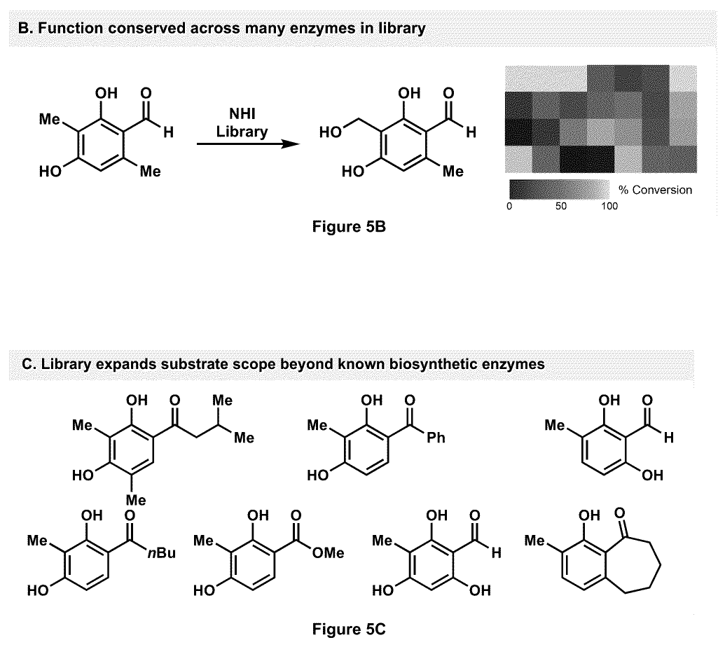

Based on this complementary substrate scope and activity, more biocatalysts related to CitB and ClaD were profiled, through analysis of the sequence space around the two enzymes. This was accomplished by generating an SSN of proteins related to CitB (FIG. 5A). SSNs enable large numbers of related protein sequences to be analyzed and sorted into clusters based on a user defined similarity score, which is calculated between all proteins in a given network.^{18, 25} Previous research has shown that SSNs can be used to effectively visualize common features within subsets of enzymes within a family^{18, 25-26}. The full network analyzed contained >40,000 sequences related to CitB and ClaD. Using a modest similarity score of 100 (E-value), it was found that CitB and ClaD clustered together with 168 additional proteins. 19 of these proteins were obtained and expressed and each was found to be active and capable of hydroxylating a model substrate, with seven of these biocatalysts providing >60% conversion of substrate to benzylic alcohol product. Testing a panel of phenol and resorcinol substrates revealed that the library provides a set of catalysts with an expanded substrate scope compared to the characterized members of this family. For example, neither CitB nor ClaD produced any detectable alcohol product in reactions with substrates shown in FIG. 5C, whereas the first-generation NHI library contained catalysts capable of selectively hydroxylating each substrate illustrated in FIG. 5C. In view of the need in the art for chemo- and site-selective catalysts for benzylic hydroxylation, a library containing >60 natural sequences from this enzyme family was built. This suite of biocatalysts provides a platform for exploring the synthetic capabilities of related catalysts and enables access to various hydroxylated compounds. In addition, this library of enzymes that naturally perform hydroxylation was transformed into an engineered library of halogenases through site-directed mutagenesis (SDM) to deliver a first-coordinate sphere at iron analogous to what is most common in non-heme iron-dependent halogenases.³⁶

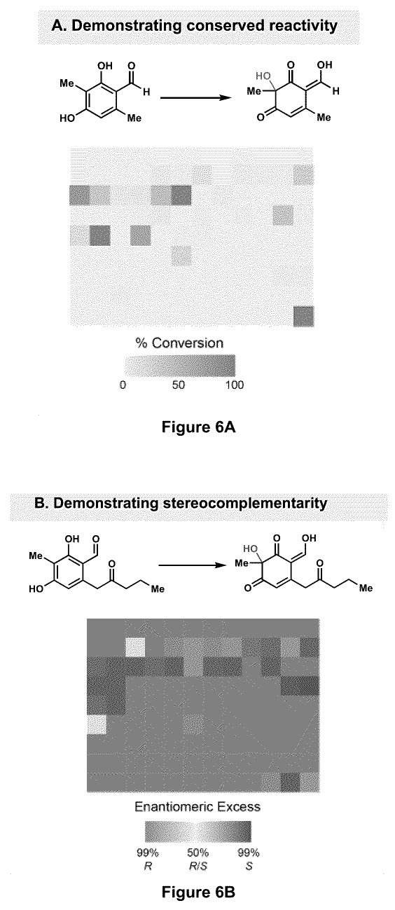

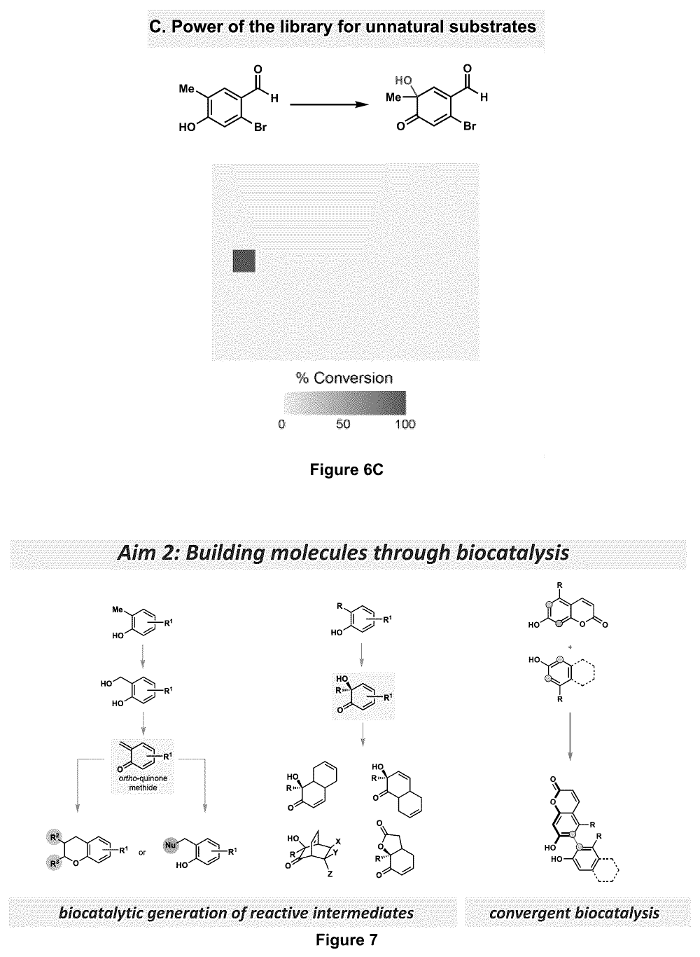

In addition to the first-generation NHI enzyme libraries for hydroxylation and halogenation, a 200+ member library of flavin-dependent monooxygenases was built and utility of this library for performing hydroxylation reactions and oxidative dearomatization reactions was demonstrated.^{5, 9} Importantly, across the library, catalysts that transform a breadth of substrates with complementary site- and stereoselectivity and in some cases divergent reactivity (aromatic hydroxylation versus dearomatization, FIG. 6) can be identified. FIG. 6A demonstrates the conservation of oxidative dearomatization activity across the first-generation Flavin-dependent monooxygenase library. FIG. 6B demonstrates the presence of stereocomplementary catalysts within the first-generation Flavin-dependent monooxygenase library. FIG. 6C demonstrates the need for substantial libraries of enzymes to capture broad substrate scope.

### Building Molecules Through Biocatalysis

High throughput construction of compound libraries can be prepared by (a) using biocatalysts to generate reactive intermediates that can be intercepted by small molecule reagents in situ, in one-pot sequences, without isolation of the intermediates and/or (b) employing biocatalysts that execute convergent reactions whereby various monomers can be cross coupled on demand. Traditionally, biocatalysis in chemical synthesis has been reserved for functional group interconversions and has not taken center stage to play a key role in assembling molecular frameworks. This limited application of biocatalysis does not capture the full potential of enzymatic synthesis.

Convergent synthetic strategies enable the efficient construction of carbon frameworks, quickly generating complexity by stitching individual building blocks together. Chemists depend on reactions that can be reliably programmed into synthetic routes, such as cross-coupling reactions, for convergent approaches.³⁷ Ideally, reactions planned for the assembly phase of a convergent synthesis are both perfectly selective and tolerate a breadth of functional groups to minimize the production of undesired products, installation of protecting groups, or unnecessary redox manipulations.³⁸ These two qualities are common in biocatalytic reactions; however, the vast majority of enzymatic transformations applied in synthesis are confined to single functional group interconversions and do not provide opportunities for convergent biocatalytic assembly of molecules.³⁹⁻⁴¹ The use of biocatalysts in retrosynthetic analysis has, therefore, largely been limited to the synthesis of small, enantioenriched building blocks or the late-stage manipulation of complex molecules, as demonstrated in the industrial syntheses of atorvastatin and sitagliptin, respectively.⁴²⁻⁴³

This missed opportunity in biocatalysis is further highlighted by the structural complexity of carbon frameworks represented in natural products assembled through total enzymatic synthesis. The most well-studied biosynthetic pathways embrace linear blueprints.⁴⁴⁻⁴⁷ Although encountered rarely, Nature does implement convergent synthetic strategies in the form of late-stage dimerization reactions; for example, the biosynthesis of gossypol, bisorbicillinol, and lomaiviticin A take place through convergent dimerization reactions.⁴⁸⁻⁵¹ Inspired by the efficiency innate to convergent biosynthetic pathways, it was recognized that biocatalysts could be deployed in complex molecule synthesis through fragment coupling reactions (FIG. 7).

Libraries of molecules using biocatalysis can be prepared using one of two distinct approaches. In the first approach, biocatalysts can be used to generate reactive intermediate which can be further transformed in the same reaction vessel (FIG. 7, left), whereas, in the second approach enzymes that mediate the coupling of two starting materials with controlled site-selectivity and, where applicable, stereoselectivity (FIG. 7, right) are relied on. To generate reactive intermediates, a number of strategies are possible, which are only limited synthetic creativity. Libraries of NIH and flavin-dependent monooxygenases can be used to generate reactive intermediates which can be transformed without isolation. For example, benzylic alcohols generated through the NHI-catalyzed hydroxylation of ortho-cresol compounds can provide access to ortho-quinone methides in situ which can be intercepted by nucleophiles or dienophiles in inverse-demand [4+2] cycloadditions. In addition, the flavin-dependent enzyme catalyzed oxidative dearomatization is expected to generate reactive dienone intermediates positioned for further transformations, including cycloaddition, acylation, and nucleophilic addition. In each case, the envisioned sequence will provide the opportunity to build onto the biocatalytically-generated intermediate with a second, modular reagent, such that each strategy can enable the synthesis of compound libraries. The strategies for interception of biocatalytically-generated intermediates have been demonstrated^{1-2, 4, 9}, including directly pairing metal-catalyzed reactions with biocatalytic reactions.⁵²⁻⁵⁴

These described sequences can be explored in a high throughput manner in reactions conducted in 96 well plates. In a typical reaction, the substrate can be combined with enzyme in the form of crude cell lysate, requisite cofactors and the reagent that will intercept the reactive intermediate. Reaction outcome can be assessed by UPLC, UPLC-MS, and/or RapidFire-MS.

In a second approach to building molecular frameworks using biocatalysis, a library of natural and engineered enzymes that carry out oxidative C—C coupling reactions can be used. For example, biaryl bond formation can be used as a model transformation, given the ubiquitous nature of biaryl scaffolds in pharmaceutical agents.⁵⁵⁻⁵⁷ Moreover, forging sterically hindered biaryl bonds presents a challenge in both reactivity and selectivity, with the need to control both the site of bond formation on each building block and the way these molecules come together in space to generate an axis of chirality with two possible atropisomers.⁵⁸⁻⁵⁹ Traditionally, sterically hindered biaryl bonds are constructed through prefunctionalization or direct oxidative coupling strategies.⁶⁰⁻⁶¹ Biocatalytic oxidative cross-coupling reactions have the potential to overcome chemoselectivity and reactivity challenges inherent to established methods by providing a paradigm with catalyst-controlled selectivity. Thus, expedited access to molecules for drug discovery can be provided. Nature has evolved catalysts for oxidative dimerization of phenolic compounds to generate biaryl natural products.^{57, 62}

An enzyme library for biaryl C—C bond formation can include wild type laccases and cytochrome P450s either known to naturally carry out this chemistry or that are proximal in sequence space to enzymes with this desired function. These enzymes can be obtained using either E. coli or Pichia pastoris as a heterologous expression hosts. Reactions can be conducted in 96 and 384 well plates screening enzyme libraries against large panels of aromatic and heteroaromatic substrates. Reaction outcome can be assessed by UPLC, UPLC-MS, and/or RapidFire-MS monitoring for cross coupling as well as dimerization of each substrate. Reactivity and selectivity screening of the first-generation library can inform the design of a second-generation library through expansion of the wild type catalysts available and protein engineering to tune substrate scope or selectivity of efficient catalysts identified in the initial screening effort. Thus, a library of enzymes capable of cross coupling a variety of substrate classes to afford sufficient quantities of compound for initial biological assays can be provided.

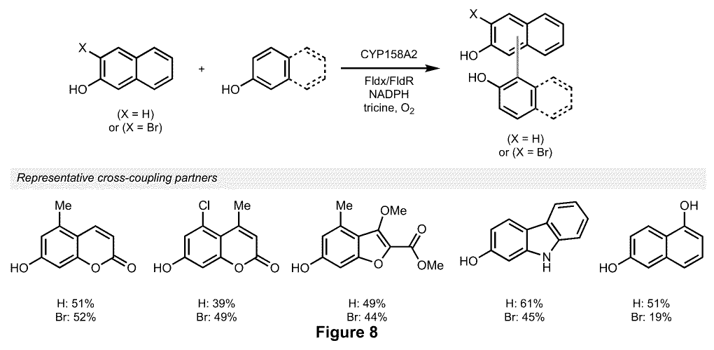

As shown in the examples, biocatalytic construction of molecules has been done by (a) using biocatalysts to generate reactive intermediates that are then intercepted by small molecule reagents in situ, and (b) employing biocatalysts that execute convergent reactions whereby various monomers can be cross coupled on demand. With regard to (a) the ortho-quinone methide chemoenzymatic sequence² was demonstrated for the target-oriented synthesis of a number of natural product families. This strategy is expected to translate to high throughput library generation as it has been observed that a breadth of nucleophiles and cycloaddition partners are compatible with this chemoenzymatic sequence. Similarly, transformations of the reactive dienone products generated through flavin-dependent enzyme-catalyzed dearomatization have been successfully demonstrated,^{4-5, 9} enabling a number of reactions beyond those depicted in FIG. 7 to be possible in this platform. With regard to (b) biocatalytic C—C biaryl bond formation, was demonstrated with a panel of non-native substrates, the results suggest that fungal P450s⁶³ have some degree of substrate promiscuity in their inherent catalytic biaryl-bond forming chemistries and can provide hundreds of milligrams of enantio-enriched tetra-ortho-substituted biaryl. Biocatalytic dimerization and cross coupling of non-native substrates with KtnC resulted in the exclusive formation of 8,8′-products, whereas dimerization with a related enzyme, DesC, lead to the formation of 6,8′-products. The use of a set of bacterial P450s was also demonstrated (FIG. 8). For example, using CYP158A2 as a catalyst, the cross coupling of 2-naphthol or 3-bromo-2-naphthol to a range of phenolic compounds with catalyst-controlled site-selectivity was achieved.

Thus, a variety of routes for the biocatalytic generation of reactive intermediates can be provided and the transformation that can be readily coupled with these biocatalytic conditions can be studied. Further, a library of enzymes (e.g., P450s and others) that can cross couple with catalyst-controlled site- and stereoselectivity can be provided. Access to panels of compounds on a scale to enable biological assays, and this approach to building compound libraries will invite synthetic chemists into the world of biocatalysis.

Throughout this specification and the claims which follow, unless the context requires otherwise, the word “comprise” and variations such as “comprises” and “comprising’ will be understood to imply the inclusion of a stated integer or step or group of integers or steps but not the exclusion of any other integer or step or group of integers or steps.

It should be understood that when describing a range of values, the characteristic being described could be an individual value found within the range. For example, “a pH from about pH 4 to about pH 6” could be, but is not limited to, pH 4, 4.2, 4.6, 5.1, 5.5, etc. and any value in between such values. Additionally, “a pH from about pH 4 to about pH 6,” should not be construed to mean that the pH of a formulation in questions varies 2 pH units in the range from pH 4 to pH 6, but rather a value may be chosen in that range for the pH of the formulation, and the pH remains buffered at about that pH.

When the term “about” is used, it means that the recited number plus or minus 5%, 10%, 15% or more of that recited number. The actual variation intended is determinable from the context.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

### Methods of the Disclosure

Disclosed herein is a method for synthesizing organic compounds comprising: separately admixing a first reactant and an aqueous solvent with each biocatalyst in a library of biocatalysts to provide a library of product admixtures, wherein the admixing occurs under sustainable reaction conditions, and each product admixture comprises: (i) a first product formed from a chemical reaction between the first reactant and each biocatalyst, (ii) the aqueous solvent, and (iii) the biocatalyst. Thus, the methods disclosed herein allow multiple first reactants each to be admixed with multiple biocatalysts in the library of biocatalysts to produce a diverse set of product compounds.

In some embodiments, each biocatalyst in the library of biocatalysts is admixed with the first reactant simultaneously, or substantially simultaneously (e.g., all of the first reactants are admixed with their respective biocatalyst from the library of biocatalysts within about 1 second to about 1 minute of each other). In some cases, each biocatalyst in the library of biocatalysts is admixed with the first reactant in a non-simultaneous manner. For example, each first reactant can be admixed with its respective biocatalyst in the library of biocatalysts at a different time period.

The first reactant can be any organic compound (e.g., small molecule) capable of undergoing a chemical transformation via enzymatic catalysis. Suitable first reactants for the methods disclosed herein have been disclosed supra. In some cases, the first reactant is a small molecule drug or a precursor to a small molecule drug.

The aqueous solvent can be any biologically compatible solution that contains water. Contemplated aqueous solvents include buffers, such as acetate, glutamate, citrate, succinate, tartrate, fumarate, maleate, histidine, phosphate, 2-(N-morpholino)ethanesulfonate, potassium phosphate, acetic acid/sodium acetate, citric acid/sodium citrate, succinic acid/sodium succinate, tartaric acid/sodium tartrate, histidine/histidine HCl, glycine, Tris, phosphate, aspartate, and combinations thereof. Several factors are typically considered when choosing a buffer. For example, the buffer species and its concentration should be defined based on its pKa and the desired pH of the reaction. Also important is to ensure that the buffer is compatible with the biocatalyst, first reactant (e.g., drug), and does not catalyze any degradation reactions. The buffer may be present in any amount suitable to maintain the pH of the formulation at a predetermined level. The buffer may be present at a concentration between about 0.1 mM and about 1000 mM (1 M), or between about 5 mM and about 200 mM, or between about 5 mM to about 100 mM, or between about 10 mM and 50 about mM. Suitable buffer concentrations encompass concentrations of about 200 mM or less. In some embodiments, the buffer in the formulation is present in a concentration of about 190 mM, about 180 mM, about 170 mM, about 160 mM, about 150 mM, about 140 mM, about 130 mM, about 120 mM, about 110 mM, about 100 mM, about 80 mM, about 70 mM, about 60 mM, about 50 mM, about 40 mM, about 30 mM, about 20 mM, about 10 mM or about 5 mM. In some embodiments, the concentration of the buffer is at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, or 900 mM. In some embodiments, the concentration of the buffer is between 1, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, or 90 mM and 100 mM. In some embodiments, the concentration of the buffer is between 5, 6, 7, 8, 9, 10, 11, 12, 13, 14, 15, 16, 17, 18, 19, 20, 30, or 40 mM and 50 mM. In some embodiments, the concentration of the buffer is about 10 mM.

Accordingly, in some embodiments, the pH of the aqueous solvent is in a range of about 3 to about 8 (e.g., about 3.0, 3.5, 4.0, 4.5, 5.0, 5.5, 6.0, 6.5, 7.0, 7.5 or 8.0). In some embodiments, the pH of the aqueous solvent is in a range of about 4.0 to about 8.0, or about 5.0 to about 8.0, or about 6.0 to about 7.5. In some embodiments, the pH of the aqueous solvent is at physiological pH (e.g., pH 7.4).

The library of product admixtures is the compilation of the reaction solutions that result from separately admixing the first reactant and each biocatalyst in the library of biocatalyst in the aqueous solvent. Each product admixture includes the aqueous solvent, the biocatalyst, the first product, and byproducts (if any) that are produced as a result of the reaction between the first reactant and its respective biocatalyst.

The admixing steps described herein occur under sustainable reaction conditions. As used herein, the term “sustainable reaction conditions” refers to reaction conditions that minimize or eliminate the use and generation of substances that are hazardous to the environment and/or a biological system, that maintain the integrity of the biocatalysts (e.g., do not cause the biocatalysts to under physical or chemical degradation, aggregation, or misfolding), and that generate benign byproducts (if any byproducts are generated). Thus, sustainable reaction conditions do not use toxic or hazardous reagents (e.g., heavy metals and environmentally detrimental solvents). The ability of the methods disclosed herein to be conducted using sustainable reaction conditions is advantageous as they allow each product admixture to be directly used in a subsequent chemical reaction or in biological assay, for example, without requiring isolation or purification of the first product.

Accordingly, the methods disclosed herein can further include admixing a second reactant in situ with one or more product admixtures in the library of product admixtures, wherein the second reactant reacts with the first product in the one or more product admixtures to form a second product. Alternatively, the methods disclosed herein can further include subjecting one or more of the first products to one or more biological assays without isolating the one or more first products from the one or more product admixtures. Thus, the methods disclosed herein allow the first products to advantageously be used in a subsequent chemical reaction or in a biological assay without isolating or purifying the first products.

Suitable biocatalysts for the methods disclosed herein have been disclosed supra. In embodiments, at least one biocatalyst in the library of biocatalysts is a flavin-dependent monooxygenase, a non-heme iron-dependent dioxygenase, a methyltransferase, a trifluoromethyltransferase, an acetyltransferase, a hydroxylase, a halogenase, or cytochrome P450. In some embodiments, each biocatalyst in the library of biocatalysts can be a wild-type enzyme or an engineered enzyme. In some embodiments, at least one biocatalyst in the library of biocatalysts is a wild-type enzyme. In various embodiments, at least one biocatalyst in the library of biocatalysts is an engineered enzyme. In some cases, at least one biocatalyst in the library of biocatalysts is a wild-type enzyme and at least one biocatalyst in the library of biocatalysts is an engineered enzyme. In some cases, one or more of the biocatalysts in the library of biocatalysts performs a site-selection chemical reaction, a stereoselective chemical reaction, a chemoselective chemical reaction, or a combination thereof in varying levels of selectivity. In some cases, one or more of the biocatalysts in the library of biocatalysts performs a site-selection chemical reaction. In some cases, one or more of the biocatalysts in the library of biocatalysts performs a stereoselective chemical reaction. In some cases, one or more of the biocatalysts in the library of biocatalysts performs a chemoselective chemical reaction.

In some embodiments, the first reactant is admixed with a biocatalyst to undergo a functional group transformation. Suitable functional group transformation reactions have been described supra. In some embodiments, the functional group transformation is a hydroxylation, halogenation, epoxidation, a C—H insertion, or a dehydrogenation. In embodiments, the functional group transformation is an alkyl hydroxylation, an aryl hydroxylation, an alkyl halogenation, or an aryl halogenation.

In some embodiments, the first reactant is admixed with a biocatalyst to undergo a carbon-carbon bond forming reaction. Suitable carbon-carbon bond forming reactions have been described supra. In some embodiments, the carbon-carbon bond forming reaction is an alkylation, an arylation, or a cyclization. In various embodiments, the alkylation is a methylation or a fluoroalkylation. In some cases, the arylation is biaryl bond forming reaction.

The library of biocatalysts can be prepared by constructing one or more phylogenetic trees, one or more sequence similarity networks (SSNs), one or more variational autoencoder (VAE) latent space analyses, or a combination thereof of from sequence data, by assessing the sequence relationship with enzymes of known function, and selecting biocatalysts for inclusion in the curated library based on sequence and sequence-function relationships.

Further disclosed herein is a method of diversifying a biologically active molecule. This method includes separately admixing the biologically active molecule and an aqueous solvent with each biocatalyst in a library of biocatalysts to provide a library of biologically active product admixtures, wherein the admixing occurs under sustainable reaction conditions, and each biologically active product admixture comprises: (i) a first biological product formed from a chemical reaction between the biologically active molecule and each biocatalyst, (ii) the aqueous solvent, and (iii) the biocatalyst.

The following examples are provided for illustration and are not intended to limit the scope of the invention.

### EXAMPLES

### Standard Procedure for Growing Plates of Biocatalyst Library:

96-well plates containing 500 μL of LB media and the appropriate antibiotic were inoculated with glycerol stocks containing transformed E. coli cells, with each well corresponding to a different biocatalyst. The plates were incubated at 37° C. until the cultures reach an optical density of 0.8, after which enzyme overexpression was induced with IPTG (0.5 mM). After 14 hours, plates were centrifuged and the supernatant was discarded. The resulting whole-cell pellets which contain enzyme can subsequently used in biocatalytic reactions.

Standard Reaction Screening with NHI Library:

200 μL of a mixture containing water, TES buffer (pH 7.5, 50 mM), NaAsc (4 mM), α-KG (4 mM), Fe₂SO₄ (0.2 mM), and substrate (2.5 mM) was dosed into each well containing cell pellet with overexpressed enzyme. The cell pellet was resuspended in the mixture and 10 μL of toluene was added to each well. Reaction plates are shaken at 200 rpm at 30° C. for 1 to 3 hours. Then the reactions were quenched with 3× volumes of acetonitrile or methanol. Plates were then centrifuged to remove cellular and biological debris, and the supernatant was filtered and submitted for analysis by UPLC-UV-MS.

### Example 1

### Preparation of α-KG-Dependent NHI Oxygenases Library for Late-Stage Functionalization Platform

Two dioxygenases associated with natural product biosynthesis, CitB and ClaD, were evaluated for substrate promiscuity and scalability. To obtain each dioxygenase biocatalyst, an expression plasmid was constructed using a synthetic codon-optimized citB or claD gene. Plasmids were commercially ordered with the input of the published gene sequence, or were constructed by PCR amplification from commercially ordered DNA. Chemically competent E. Coli cells were transformed through heat shock with the plasmid DNA encoding for the desired enzyme. Subsequent overexpression in E. coli provided large quantities of each biocatalyst (60-150 mg/L). To achieve overexpression, transformed E. coli cells were cultured in 0.5 liters of TB media, and expression of the enzyme gene was induced with the addition of isopropyl β-D-1-thiogalactopyranoside (IPTG).

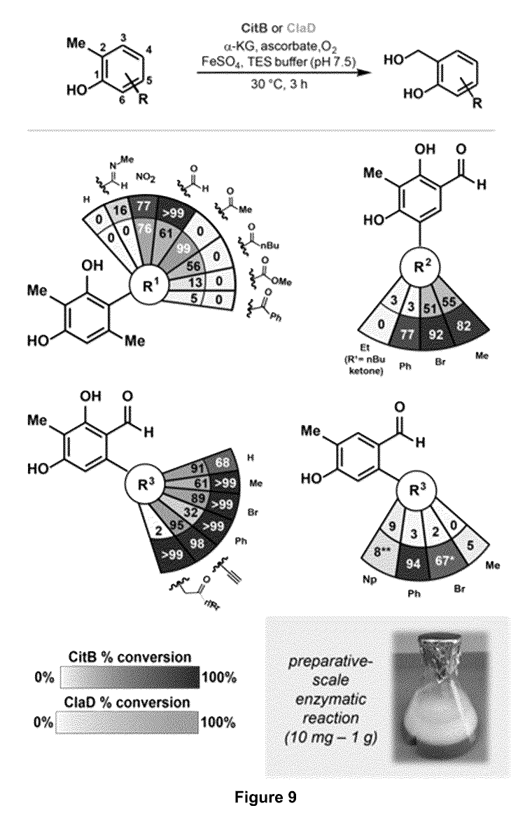

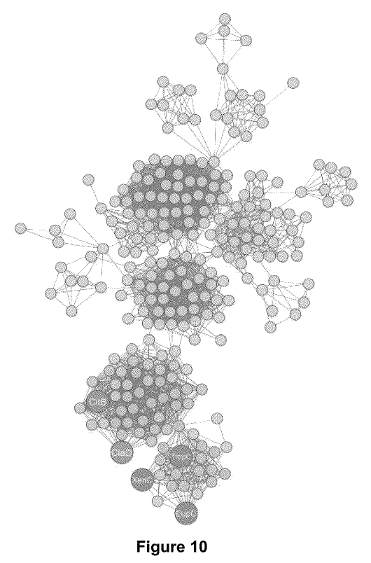

For evaluation of CitB reactivity, a panel of substrates was subjected to 0.4 mol % enzyme in the presence of NaAsc (1.6 equiv), α-KG (1.6 equiv) and FeSO₄ (8 mol %). Reactions were conducted in 50 mM aqueous TES buffer (pH 7.5) at 30° C. for 1 to 3 hours. CitB mediated the oxidation of a range of substrates, as observed by UHPLC-UV-MS analysis (FIG. 9). The activity of ClaD was similarly evaluated with a range of resorcinol and phenol substrates. These experiments revealed some complementarity in the substrate scope of the two enzymes. In many cases where oxidation by one enzyme was not observed, the other was shown to be capable of oxidation.

A sequence similarity network (SSN) of proteins related to CitB was generated using the EFI—Enzyme Similarity Tool. The SSN analyzed contained >40,000 sequences related to CitB and ClaD. Using a modest similarity score of 100 (E-value), it was found that CitB and ClaD clustered together with 168 additional proteins. 19 of these proteins were obtained and expressed following the same procedure for expression of CitB and ClaD. All 19 were found to be active and capable of hydroxylating a model substrate (FIG. 5B), with seven of these biocatalysts providing >60% conversion of substrate to benzylic alcohol product: NHI_1, NHI_2, NHI_6, NHI_10, NHI_14, NHI_15, and NHI_17. Testing a panel of phenol and resorcinol substrates under identical conditions to CitB/ClaD reactions revealed that the library of approximately 60 wild type enzymes provides a set of catalysts with an expanded substrate scope compared to the characterized members of this family. For example, neither CitB nor ClaD produced any detectable alcohol product in reactions with substrates shown in FIG. 5C, whereas the first-generation NHI library contained catalysts capable of selectively hydroxylating each substrate illustrated in FIG. 5C. A library containing >60 natural sequences has been built from this enzyme family. This initial suite of biocatalysts provides a platform for exploring the synthetic capabilities of related catalysts and enables access to various hydroxylated compounds.

Thus, this example demonstrates the building of a library of catalysts with an expanded substrate scope compared to that of the characterized members of this family.

### Example 2

### Target Oriented Synthesis of Natural Product Families

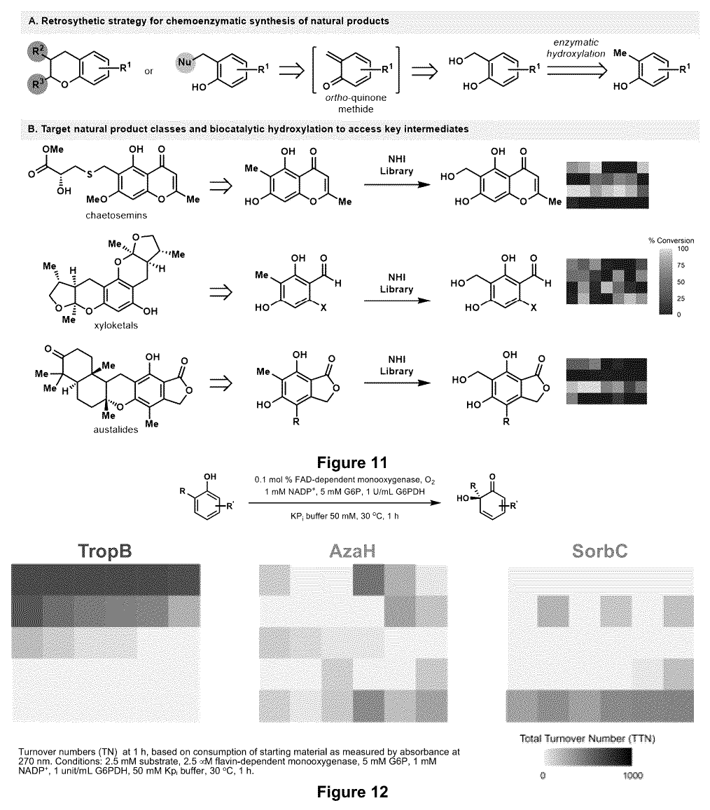

Toward the biocatalytic construction of molecules, preliminary experiments were to support the feasibility of chemoenzymatic strategies employing NHI-dependent oxygenases. Toward the biocatalytic generation of reactive intermediates and chemoenzymatic strategies to intercept these fleeting intermediates without the need for isolation, the feasibility was demonstrated of the proposed ortho-quinone methide chemoenzymatic sequence, and this strategy was applied in the target-oriented synthesis of a number of natural product families, including those outlined in FIG. 11. This strategy will translate to high throughput library generation as has been a breadth of nucleophiles and cycloaddition partners were observed to be compatible with this chemoenzymatic sequence.

### Example 3

### Use of Flavin-Dependent Monooxygenases for Hydroxylation and Oxidative Dearomatization

In addition to a first-generation NHI enzyme libraries for hydroxylation and halogenation, a 200+ member library of flavin-dependent monooxygenases was built, and the utility of this library for performing hydroxylation reactions and oxidative dearomatization reactions was demonstrated. Importantly, across the library, catalysts that transform a breadth of substrates with complementary site- and stereoselectivity were identified, and in some cases divergent reactivity (aromatic hydroxylation versus dearomatization, FIG. 12).

In addition, the library of flavin-dependent monooxygenases was demonstrated to possesses conserved function, stereocomplementary catalysts, and has utility for target oriented synthesis (FIG. 6). FIG. 6 shows the activity across the first-generation library of flavin-dependent monooxygenases. FIG. 6A shows the conservation of oxidative dearomatization activity, FIG. 6B shows a profiling of the stereoselectivity across the library of flavin-dependent monooxygenases to illustrate the stereo-divergent nature of the enzymes in the library, and FIG. 6C demonstrates the utility of flavin-dependent monooxygenase library for target oriented synthesis.

### Example 4

### Biocatalytic C—C Bond Formation

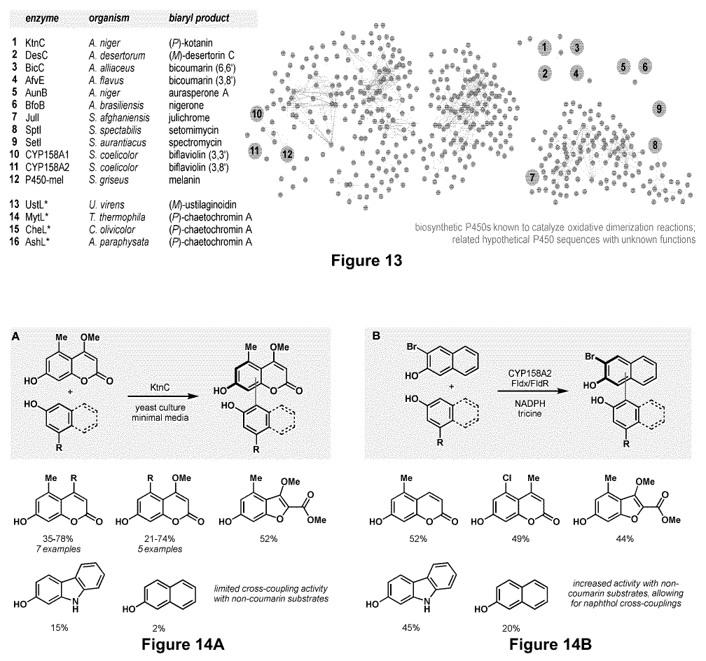

A first-generation library of enzymes capable of biaryl C—C bond formation has been assembled. See, e.g., FIG. 6. The first-generation library consists of wild type cytochrome P450s either known to naturally carry out this chemistry or that are proximal in sequence space to enzymes with this desired function (FIG. 13). These enzymes were obtained using either E. coli or Pichia pastoris as a heterologous expression host. Reactions were conducted in 96 and 384 well plates screening enzyme libraries against large panels of aromatic and heteroaromatic substrates. Reaction outcome was assessed by UPLC, UPLC-MS, and/or RapidFire-MS monitoring for cross coupling as well as dimerization of each substrate.

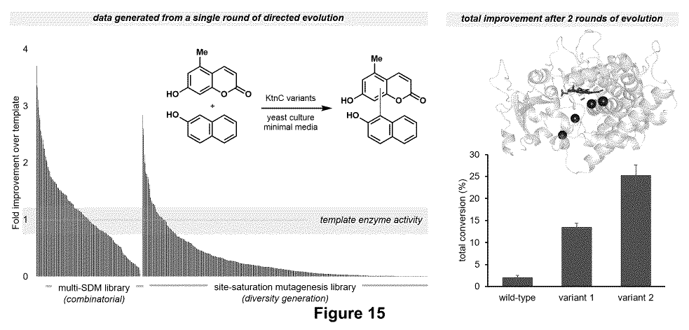

For biocatalytic C—C biaryl bond formation, the preliminary experiments with a panel of non-native substrates suggested that fungal P450s have some degree of substrate promiscuity in their inherent catalytic biaryl-bond forming chemistries and can provide hundreds of milligrams of enantio-enriched tetra-ortho-substituted biaryl products (FIG. 14A). Biocatalytic dimerization and cross coupling of non-native substrates with KtnC resulted in the exclusive formation of 8,8′-products, whereas dimerization with a related enzyme, DesC, lead to the formation of 6,8′-products. More spectacular results were obtained with a set of bacterial P450s. For example, using CYP158A2 as a catalyst, the cross coupling of 2-naphthol or 3-bromo-2-naphthol to a range of phenolic compounds with catalyst-controlled site-selectivity was achieved (FIG. 14B).

Toward the development of robust catalysts for convergent biocatalysis, it is anticipated that a medium-sized library of wild type sequences will be required in addition to protein engineering to achieve a panel of catalysts suitable for generation of a breadth of compounds. Proteins can be engineered using site-saturation mutagenesis, combinatorial site-saturation mutagenesis as well as error-prone PCR to generate libraries. These libraries have demonstrated expanded substrate scope, improved yields and enhanced selectivity (FIG. 15).

The foregoing description is given for clearness of understanding only, and no unnecessary limitations should be understood therefrom, as modifications within the scope of the invention may be apparent to those having ordinary skill in the art.

All patents, publications and references cited herein are hereby fully incorporated by reference. In case of conflict between the present disclosure and incorporated patents, publications and references, the present disclosure should control.

The following paragraphs provide the references cited herein.
1. Pyser, J. B.; Dockrey, S. A. B.; Benitez, A. R.; Joyce, L. A.; Wiscons, R. A.; Smith, J. L.; Narayan, A. R. H., Stereodivergent, Chemoenzymatic Synthesis of Azaphilone Natural Products. Journal of the American Chemical Society 2019, 141 (46), 18551-18559.
2. Doyon, T. J.; Perkins, J. C.; Dockrey, S. A. B.; Romero, E. O.; Skinner, K. C.; Zimmerman, P. M.; Narayan, A. R. H., Chemoenzymatic o-Quinone Methide Formation. Journal of the American Chemical Society 2019, 141 (51), 20269-20277.
3. Chun, S. W.; Narayan, A. R. H., Biocatalytic Synthesis of alpha-Amino Ketones. Synlett 2019, 30 (11), 1269-1274.
4. Dockrey, S. A. B.; Suh, C. E.; Benitez, A. R.; Wymore, T.; Brooks, C. L.; Narayan, A. R. H., Positioning-Group-Enabled Biocatalytic Oxidative Dearomatization. Acs Central Science 2019, 5 (6), 1010-1016.
5. Pyser, J. B., Baker Dockrey, S. A., Joyce, L. A., Rodríguez Benítez, A. Wiscons, R. A. Narayan, A. R. H., Stereodivergent, chemoenzymatic synthesis of azaphilone natural products. J. Am. Chem. Soc. 2019, Manuscript under revision.
6. Dockrey, S. A. B.; Doyon, T. J.; Perkins, J. C.; Narayan, A. R. H., Whole-cell biocatalysis platform for gram-scale oxidative dearomatization of phenols. Chemical Biology & Drug Design 2019, 93 (6), 1207-1213.
7. Benitez, A. R.; Tweedy, S. E.; Dockrey, S. A. B.; Lukowski, A. L.; Wymore, T.; Khare, D.; Brooks, C. L.; Palfey, B. A.; Smith, J. L.; Narayan, A. R. H., Structural Basis for Selectivity in Flavin-Dependent Monooxygenase-Catalyzed Oxidative Dearomatization. Acs Catalysis 2019, 9 (4), 3633-3640.
8. Lukowski, A. L.; Denomme, N.; Hinze, M. E.; Hall, S.; Isom, L. L.; Narayan, A. R. H., Biocatalytic Detoxification of Paralytic Shellfish Toxins. ACS Chem. Biol. 2019, 14 (5), 941-948.
9. Baker Dockrey, S. A.; Lukowski, A. L.; Becker, M. R.; Narayan, A. R. H., Biocatalytic site- and enantioselective oxidative dearomatization of phenols. Nat Chem 2018, 10 (2), 119-125.
10. Lukowski, A. L.; Ellinwood, D. C.; Hinze, M. E.; DeLuca, R. J.; Du Bois, J.; Hall, S.; Narayan, A. R. H., C—H Hydroxylation in Paralytic Shellfish Toxin Biosynthesis. Journal of the American Chemical Society 2018, 140 (37), 11863-11869.
11. Chun, S. W.; Hinze, M. E.; Skiba, M. A.; Narayan, A. R. H., Chemistry of a Unique Polyketide-like Synthase. J. Am. Chem. Soc. 2018, 140 (7), 2430-2433.
12. Newman, D. J.; Cragg, G. M., Natural products as sources of new drugs over the last 25 years. J. Nat. Prod. 2007, 70 (3), 461-477.
13. Truppo, M. D., Biocatalysis in the Pharmaceutical Industry: The Need for Speed. Acs Medicinal Chemistry Letters 2017, 8 (5), 476-480.
14. Sheldon, R. A.; Woodley, J. M., Role of Biocatalysis in Sustainable Chemistry. Chem. Rev. 2018, 118 (2), 801-838.
15. Sheldon, R. A.; Brady, D., The limits to biocatalysis: pushing the envelope. Chem. Commun. 2018, 54 (48), 6088-6104.
16. Bornscheuer, U. T., Biocatalysis: Successfully Crossing Boundaries. Angew. Chem.-Int. Edit. 2016, 55 (14), 4372-4373.
17. Tomás, R. A. F.; Bordado, J. C. M.; Gomes, J. F. P., p-Xylene Oxidation to Terephthalic Acid: A Literature Review Oriented toward Process Optimization and Development. Chem. Rev. 2013, 113 (10), 7421-7469.
18. Gerlt, J. A.; Bouvier, J. T.; Davidson, D. B.; Imker, H. J.; Sadkhin, B.; Slater, D. R.; Whalen, K. L., Enzyme Function Initiative-Enzyme Similarity Tool (EFI-EST): A web tool for generating protein sequence similarity networks. Biochimica et Biophysica Acta (BBA)—Proteins and Proteomics 2015, 1854 (8), 1019-1037.
19. Ryle, M. J.; Hausinger, R. P., Non-heme iron oxygenases. Current Opinion in Chemical Biology 2002, 6 (2), 193-201.
20. Krebs, C.; Fujimori, D. G.; Walsh, C. T.; Bollinger, J. M., Non-heme Fe(IV)-oxo intermediates. Accounts Chem. Res. 2007, 40 (7), 484-492.
21. Martinez, S.; Hausinger, R. P., Catalytic Mechanisms of Fe(II)- and 2-Oxoglutarate-dependent Oxygenases. J. Biol. Chem. 2015, 290 (34), 20702-20711.
22. Davison, J.; al Fahad, A.; Cai, M. H.; Song, Z. S.; Yehia, S. Y.; Lazarus, C. M.; Bailey, A. M.; Simpson, T. J.; Cox, R. J., Genetic, molecular, and biochemical basis of fungal tropolone biosynthesis. Proc. Natl. Acad. Sci. U.S.A 2012, 109 (20), 7642-7647.
23. He, Y.; Cox, R. J., The molecular steps of citrinin biosynthesis in fungi. Chemical Science 2016, 7 (3), 2119-2127.
24. Fan, J.; Liao, G.; Kindinger, F.; Ludwig-Radtke, L.; Yin, W. B.; Li, S. M., Peniphenone and Penilactone Formation in Penicillium crustosum via 1,4-Michael Additions of ortho-Quinone Methide from Hydroxyclavatol to gamma-Butyrolactones from Crustosic Acid. J. Am. Chem. Soc. 2019, 141 (10), 4225-4229.
25. Cheng, S.; Karkar, S.; Bapteste, E.; Yee, N.; Falkowski, P.; Bhattacharya, D., Sequence similarity network reveals the imprints of major diversification events in the evolution of microbial life. Frontiers in Ecology and Evolution 2014, 2 (72).
26. Atkinson, H. J.; Morris, J. H.; Ferrin, T. E.; Babbitt, P. C., Using Sequence Similarity Networks for Visualization of Relationships Across Diverse Protein Superfamilies. PLOS One 2009, 4 (2), e4345.
27. Ding, X. Q.; Zou, Z. T.; Brooks, C. L., Deciphering protein evolution and fitness landscapes with latent space models. Nature Communications 2019, 10, 13.
28. Belli, A.; Giordano, C.; Citterio, A., Benzyl Acetates by Acetoxylation of Methylbenzenes using Peroxydisulfate as Oxidizing Agent. Synthesis 1980, 1980 (06), 477-479.
29. Huang, X. Y.; Groves, J. T., Oxygen Activation and Radical Transformations in Heme Proteins and Metalloporphyrins. Chem. Rev. 2018, 118 (5), 2491-2553.
30. Baciocchi, E.; Mandolini, L.; Rol, C., Oxidation by metal ions. 6. Intramolecular selectivity in the side-chain oxidation of p-ethyltoluene and isodurene by cobalt(III), cerium(IV), and manganese(III). The Journal of Organic Chemistry 1980, 45 (19), 3906-3909.
31. Simmons, E. M.; Hartwig, J. F., Catalytic functionalization of unactivated primary C—H bonds directed by an alcohol. Nature 2012, 483, 70.
32. Zhang, B.; Zhu, S.-F.; Zhou, Q.-L., Copper-catalyzed benzylic oxidation of C(sp3)-H bonds. Tetrahedron 2013, 69 (8), 2033-2037.
33. Rebelo, S. L. H.; Simões, M. M. Q.; Neves, M. G. P. M. S.; Silva, A. M. S.; Tagliatesta, P.; Cavaleiro, J. A. S., Oxidation of bicyclic arenes with hydrogen peroxide catalysed by Mn(III) porphyrins. Journal of Molecular Catalysis A: Chemical 2005, 232 (1), 135-142.
34. Wang, F.; Xu, J.; Liao, S.-j., One-step heterogeneously catalytic oxidation of o-cresol by oxygen to salicylaldehyde. Chemical Communications 2002, (6), 626-627.
35. Fisher, B. F.; Snodgrass, H. M.; Jones, K. A.; Andorfer, M. C.; Lewis, J. C., Site-Selective C—H Halogenation Using Flavin-Dependent Halogenases Identified via Family-Wide Activity Profiling. Acs Central Science 2019, 5 (11), 1844-1856.
36. Lewis, J. C.; Coelho, P. S.; Arnold, F. H., Enzymatic functionalization of carbon-hydrogen bonds. Chemical Society Reviews 2011, 40 (4), 2003-2021.
37. Yin; Liebscher, J., Carbon-carbon coupling reactions catalyzed by heterogeneous palladium catalysts. Chem. Rev. 2007, 107 (1), 133-173.
38. Gaich, T.; Baran, P. S., Aiming for the ideal synthesis. J. Org. Chem. 2010, 75 (14), 4657-4673.
39. Bornscheuer, U. T.; Huisman, G. W.; Kazlauskas, R. J.; Lutz, S.; Moore, J. C.; Robins, K., Engineering the third wave of biocatalysis. Nature 2012, 485 (7397), 185-194.
40. Turner, N. J.; O'Reilly, E., Biocatalytic retrosynthesis. Nat. Chem. Biol. 2013, 9 (5), 285-288.
41. Prier, C. K.; Kosjek, B., Recent preparative applications of redox enzymes. Curr. Opin. Chem. Biol. 2019, 49, 105-112.
42. Ma, S. K.; Gruber, J.; Davis, C.; Newman, L.; Gray, D.; Wang, A.; Grate, J.; Huisman, G. W.; Sheldon, R. A., A green-by-design biocatalytic process for atorvastatin intermediate. Green Chemistry 2010, 12 (1), 81-86.
43. Savile, C. K.; Janey, J. M.; Mundorff, E. C.; Moore, J. C.; Tam, S.; Jarvis, W. R.; Colbeck, J. C.; Krebber, A.; Fleitz, F. J.; Brands, J.; Devine, P. N.; Huisman, G. W.; Hughes, G. J., Biocatalytic asymmetric synthesis of chiral amines from ketones applied to sitagliptin manufacture. Science 2010, 329 (5989), 305-309.
44. Lange, B. M.; Rujan, T.; Martin, W.; Croteau, R., Isoprenoid biosynthesis: the evolution of two ancient and distinct pathways across genomes. Proc. Natl. Acad. Sci. 2000, 97 (24), 13172.
45. Staunton, J.; Weissman, K. J., Polyketide biosynthesis: a millennium review. Nat. Prod. Rep. 2001, 18 (4), 380-416.
46. Maeda, H.; Dudareva, N., The shikimate pathway and aromatic amino acid biosynthesis in plants. Annu. Rev. Plant Biol. 2012, 63 (1), 73-105.
47. Tang, M.-C.; Zou, Y.; Watanabe, K.; Walsh, C. T.; Tang, Y., Oxidative cyclization in natural product biosynthesis. Chem. REv. 2017, 117 (8), 5226-5333.
48. Effenberger, I.; Zhang, B.; Li, L.; Wang, Q.; Liu, Y.; Klaiber, I.; Pfannstiel, J.; Wang, Q.; Schaller, A., Dirigent proteins from cotton (Gossypium sp.) for the atropselective synthesis of gossypol. Angew. Chem. Int. Ed. 2015, 54 (49), 14660-14663.
49. Abe, N.; Arakawa, T.; Yamamoto, K.; Hirota, A., Biosynthesis of bisorbicillinoid in Trichoderma sp. USF-2690; evidence for the biosynthetic pathway, via sorbicillinol, of sorbicillin, bisorbicillinol, bisorbibutenolide, and . . . . Biosci. Biotech. Bioch. 2002, 66 (10), 2090-2099.
50. He, H.; Ding, W.-D.; Bernan, V. S.; Richardson, A. D.; Ireland, C. M.; Greenstein, M.; Ellestad, G. A.; Carter, G. T., Lomaiviticins A and B, potent antitumor antibiotics from Micromonospora lomaivitiensis. J. Am. Chem. Soc. 2001, 123 (22), 5362-5363.
51. Lian, G.; Yu, B., Naturally occurring dimers from chemical perspective. Chem. Biodiversity 2010, 7 (11), 2660-2691.
52. Payne, J. T.; Butkovich, P. H.; Gu, Y. F.; Kunze, K. N.; Park, H. J.; Wang, D. S.; Lewis, J. C., Enantioselective Desymmetrization of Methylenedianilines via Enzyme-Catalyzed Remote Halogenation. J. Am. Chem. Soc. 2018, 140 (2), 546-549.
53. Dander, J. E.; Giroud, M.; Racine, S.; Darzi, E. R.; Alvizo, O.; Entwistle, D.; Garg, N. K., Chemoenzymatic conversion of amides to enantioenriched alcohols in aqueous medium. Communications Chemistry 2019, 2.
54. DeHovitz, J. S.; Loh, Y. Y.; Kautzky, J. A.; Nagao, K.; Meichan, A. J.; Yamauchi, M.; MacMillan, D. W. C.; Hyster, T. K., Static to inducibly dynamic stereocontrol: The convergent use of racemic beta-substituted ketones. Science 2020, 369 (6507), 1113-+.
55. Yoon, T. P.; Jacobsen, E. N., Privileged chiral catalysts. Science 2003, 299 (5613), 1691-1693.
56. Yet, L., Biaryls. In Privileged Structures in Drug Discovery, First ed.; John Wiley & Sons, Inc.: Hoboken, N J, 2018; pp 83-154.
57. Bringmann, G.; Gunther, C.; Ochse, M.; Schupp, O.; Tasler, S., Biaryls in nature: a multi-facetted class of stereochemically, biosynthetically, and pharmacologically intriguing secondary metabolites. In Progress in the chemistry of organic natural products, Bringmann, G.; Günther, C.; Ochse, M.; Schupp, O.; Tasler, S.; Herz, W.; Falk, H.; Kirby, G. W.; Moore, R. E., Eds. Springer Vienna: Vienna, 2001; pp 1-249.
58. Bringmann, G.; Gulder, T.; Gulder, T. A. M.; Breuning, M., Atroposelective total synthesis of axially chiral biaryl natural products. Chem. Rev. 2011, 111 (2), 563-639.
59. Bringmann, G.; Price Mortimer, A. J.; Keller, P. A.; Gresser, M. J.; Garner, J.; Breuning, M., Atroposelective synthesis of axially chiral biaryl compounds. Angew. Chem. Int. Ed. 2005, 44 (34), 5384-5427.
60. Kozlowski, M. C.; Morgan, B. J.; Linton, E. C., Total synthesis of chiral biaryl natural products by asymmetric biaryl coupling. Chem. Soc. Rev. 2009, 38 (11), 3193-3207.
61. Kočovský, P.; Vyskočil, Š.; Smrčina, M., Non-symmetrically substituted 1,1′-binaphthyls in enantioselective catalysis. Chem. Rev. 2003, 103 (8), 3213-3246.
62. Aldemir, H.; Richarz, R.; Gulder, T. A., The biocatalytic repertoire of natural biaryl formation. Angew. Chem. Int. Ed. 2014, 53 (32), 8286-8293.
63. Mazzaferro, L. S.; Huttel, W.; Fries, A.; Muller, M., Cytochrome P450-Catalyzed Regio- and Stereoselective Phenol Coupling of Fungal Natural Products. J. Am. Chem. Soc. 2015, 137 (38), 12289-12295.

1. A method for synthesizing organic compounds comprising: separately admixing a first reactant and an aqueous solvent with each biocatalyst in a library of biocatalysts to provide a library of product admixtures, wherein the admixing occurs under sustainable reaction conditions, and each product admixture comprises: (i) a first product formed from a chemical reaction between the first reactant and each biocatalyst, (ii) the aqueous solvent, and (iii) the biocatalyst.

2. The method of claim 1, further comprising admixing a second reactant in situ with one or more product admixtures in the library of product admixtures, wherein the second reactant reacts with the first product in the one or more product admixtures to form a second product.

3. The method of claim 1, further comprising subjecting one or more of the first products to one or more biological assays without isolating the one or more first products from the one or more product admixtures.

4. The method of claim 1, wherein at least one biocatalyst in the library of biocatalysts is a flavin-dependent monooxygenase, a non-heme iron-dependent dioxygenase, a methyltransferase, a trifluoromethyltransferase, an acetyltransferase, a hydroxylase, a halogenase, or cytochrome P450.

5. The method of claim 1, wherein each biocatalyst in the library of biocatalysts is a wild-type enzyme or an engineered enzyme.

6. The method of claim 1, wherein one or more of the biocatalysts in the library of biocatalysts performs a site-selection chemical reaction, a stereoselective chemical reaction, a chemoselective chemical reaction, or a combination thereof.

7. The method of claim 1, wherein each biocatalyst is admixed with each of the first reactants simultaneously.

8. The method of claim 1, wherein each biocatalyst is admixed with each of the first reactants non-simultaneously.

9. The method of claim 1, wherein the chemical reaction is a functional group transformation.

10. The method of claim 9, wherein the functional group transformation is a hydroxylation, halogenation, epoxidation, a C—H insertion, or a dehydrogenation.

11. The method of claim 10, wherein the functional group transformation is an alkyl hydroxylation, an aryl hydroxylation, an alkyl halogenation, or an aryl halogenation.

12. The method of claim 1, wherein the chemical reaction is a carbon-carbon bond forming reaction.

13. The method of claim 12, wherein the carbon-carbon bond forming reaction is an alkylation, an arylation, or a cyclization.

14. The method of claim 13, wherein the alkylation is a methylation or a fluoroalkylation.

15. The method of claim 13, wherein the arylation is biaryl bond forming reaction.

16. The method of claim 1, wherein the library of biocatalysts is prepared by constructing one or more phylogenetic trees, one or more sequence similarity networks (SSNs), one or more variational autoencoder (VAE) latent space analyses, or a combination thereof of from sequence data, by assessing a sequence relationship with enzymes of known function, and selecting biocatalysts for inclusion in the curated library based on sequence and sequence-function relationships

17. A method of diversifying a biologically active molecule comprising: separately admixing the biologically active molecule and an aqueous solvent with each biocatalyst in a library of biocatalysts to provide a library of biologically active product admixtures, wherein the admixing occurs under sustainable reaction conditions, and each biologically active product admixture comprises: (i) a first biological product formed from a chemical reaction between the biologically active molecule and each biocatalyst, (ii) the aqueous solvent, and (iii) the biocatalyst.
